(19) United States
(12) Patent Application Publication
(10) Pub. No.: US 2008/0306398 A1
(43) Pub. Date: Dec. 11, 2008

(54) BRAIN WAVE DETECTING APPARATUS

(75) Inventors: Takuya Uchiyama, Shinagawa (JP); Satoshi Sakural, Shinagawa (JP); Shigemi Kurashlma, Shinagawa (JP); Masahiro Yanagl, Shlnagawa (JP); Takashi Yuba, Shinagawa (JP); Takashi Arita, Shinagawa (JP)

Correspondence Address: STAAS & HALSEY LLP, SUITE 700, 1201 NEW YORK AVENUE, N.W., WASHINGTON, DC, 20005, US

(73) Assignee: FUJITSU COMPONENT LIMITED, TOKYO (JP)

(21) Appl. No.: 12/134,104

(22) Filed: Jun. 5, 2008

(30) Foreign Application Priority Data
Jun. 6, 2007 (JP) 2007-150990

(51) Int. Cl. A61B 5/04

(52) U.S. Cl. 600/544

(57) ABSTRACT

A brain wave detecting apparatus includes a plurality of brain wave detecting portions that are arranged on a side surface of a head and detect brain waves of the head, a selecting portion selecting one of the plurality of brain wave detecting portions on the basis of brain wave signals transmitted from the plurality of brain wave detecting portions, and a transmitting portion transmitting information about the brain wave signal transmitted from said one of the plurality of brain wave detecting portions.

### BACKGROUND OF THE INVENTION

1. Field of the Invention

The present invention generally relates to a brain wave detecting apparatus, and more particularly to a brain wave detecting apparatus having a plurality of brain wave detecting portions that detect brain waves.

2. Description of the Related Art

A diagnostic unit for brain function and a snooze restring unit equipped with a brain wave detecting apparatus have been developed. Japanese Patent Application Publication No, 10-201727 (hereinafter described as Document 1), Japanese, Utility Model Application Publication No. 5-30796 (hereinafter described as Document 2), and Japanese Patent Application Publication No. 5-262161 (hereinafter described as Document 3) disclose snooze restraining units that restrain a driver from snoozing by detecting brain wave of the driver of an automobile or a motorcycle. Japanese Patent Application Publication No. 2006-14833 (hereinafter described as Document 4) discloses a technique that uses brain wave signals to determine brain functions. This technique uses multiple brain wave signal detecting means arranged over the head of the user. The bra functions may be determined by using brain wave signals that are output by particular brain wave signal detecting means determined to have a good condition of contact with the head of the user.

The brain wave detecting apparatus used for the snooze restraining unit as disclosed in Documents 1 through 3 is worn on the head of driver. It is necessary to ensure that the brain wave detecting apparatus detects the brain wave in a good condition, when the user wears the brain wave detecting apparatus. If the brain wave detecting apparatus cannot detect the brain wave in a good condition, the user is required to wear the brain wave detecting apparatus in a different way. It is thus complicated to wear the conventional brain wave detecting apparatus. Particularly, the brain wave detecting apparatus that is in daily use, such as for snooze restraining, is expected to be worn easily.

### SUMMARY OF THE INVENTION

The present invention has been made in view of the above-mentioned circumstances and provides a brain wave detecting apparatus that can be worn easily and can detect the brain wave stably.

According to an aspect of the present invention, there is provided a brain wave detecting apparatus including: a plurality of an wave detecting portions that are arranged on a side surface of a head and detect brain waves of the head; a selecting portion selecting one of the plurality of brain wave detecting portions on the basis of brain wave signals transmitted from the plurality of brain wave detecting portions; and a transmitting portion transmitting information about the brain wave signal transmitted from said one of the plurality of brain wave detecting portions.

### BRIEF DESCRIPTION OF THE DRAWINGS

FIG. 1A is a block diagram of a brain wave detecting apparatus in accordance with a first embodiment, and FIG. 1B shows an appearance thereof;

FIG. 2 shows an appearance of the brain wave detecting apparatus worn on a head of a user;

FIG. 3 is a flowchart of an operation of a processing portion shown in FIG. 1A;

FIG. 4 is a flowchart of another operation of the processing portion;

FIGS. 5A and 5B show that the user wears a hat low over user's eyes and shallowly, respectively; and

FIGS. 6A through 6C show that the user wears the hat with a brim thereof being laterally inclined to the right and the left, respectively.

### DESCRIPTION OF THE PREFERRED EMBODIMENTS

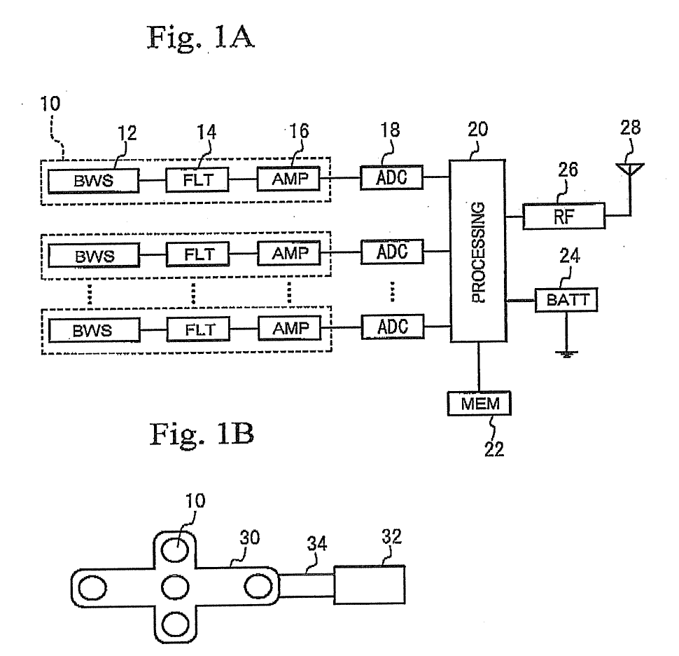

FIG. 1A is a block diagram of a brain wave detecting apparatus in accordance with a first embodiment. The bran wave detecting apparatus has a plurality of brain wave detecting portions 10, each being composed of a brain wave sensor (BWS) 12, a filter (FLT) 14, and an amplifier (AMP) 16. Further, the brain wave detecting apparatus includes multiple analog-to-digital converters (ADC) 18, a processing portion 20, a memory (MEM) 22, a battery (BATT) 24, a radio frequency (RF) circuit 26, and an antenna 28. The brain wave detecting portions 10 are coupled with the processing portion 20 via the respective ADCs 18. In each of the brain wave detecting portions 10, the brain wave sensor 12 is arranged on a head of a user such as a driver. The filter 14 eliminates noise of unnecessary frequencies from the analog brain wave signal detected by the brain wave sensor 12. The analog brain wave signal thus filtered is amplified by the amplifier 16 and is then converted to a digital brain wave signal by the ADC 18. The processing portion 20 receives the digitized brain wave signal. The processing portion 20 may be composed of a CPU (Central Processing Unit), and selects one or more brain wave detecting portions 10 to be used by referring to the brain wave signals transmitted by the plurality of the brain detecting portions 10. The processing portion 20 outputs the brain wave signals from the selected brain wave detecting portion 10 to the RF circuit 26. The RF circuit 26 modulates the RF signal by the brain wave signals. The modulated RF signal is then transmitted via the antenna 28. A battery 24 supplies electrical power to the processing portion 20 and the others.

FIG. 1B shows an appearance of the brain wave detecting apparatus in accordance with the first embodiment. A base 30 and a main printed circuit board 32 are arranged and fed to a band 34. The base 30 shaped in a cross may be composed of a flexible substrate, in which five brain wave detecting portions 10 are mounted in the present embodiment. The main printed circuit board 32 is used to mount the ADCs 18, the processing portion 20, the memory 22, the battery 24, the RF circuit 26 and the antenna 28.

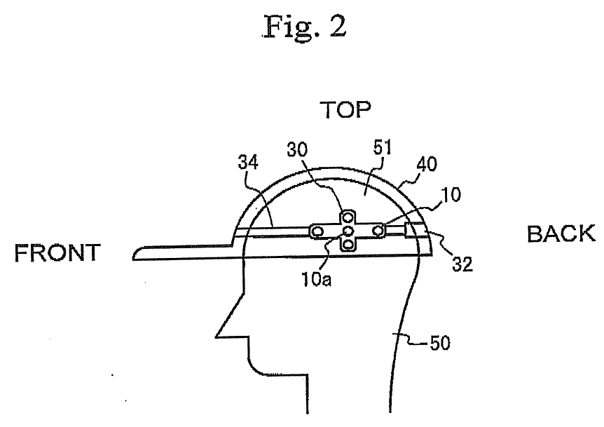

FIG. 2 shows a user 50 wearing a hat 40 in which the base 30 and the main printed circuit board 32 are fixed inside thereof with the band 34. The base 30 and the main printed circuit board 32 are shown in FIG. 2 seen through the hat 40. When the user 50 wears the hat 40, for example, the plurality of the brain wave detecting portions 10 are arranged on a side surface of a head 51. Then, the brain wave sensors 12 in the brain wave detecting portions 10 contact the scalp of the user 50. Thus, the brain wave detecting portions 10 are now capable of detecting the brain wave of the user 50.

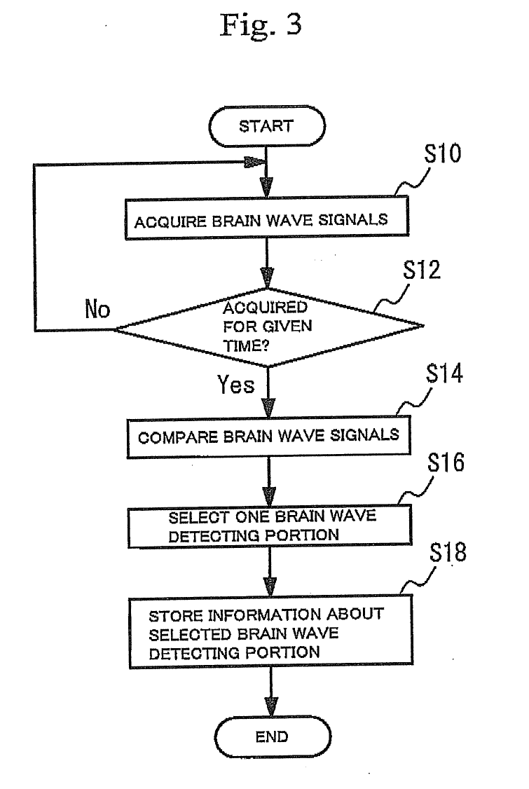

The brain wave detecting apparatus operates as follows. FIG. 3 shows a flowchart of an initialing process executed by the processing portion 20, when the user 50 wears the hat 40. Referring to FIG. 3, the processing portion 20 acquires the brain wave signals transmitted from the multiple (five) brain wave detecting portions 10 (step S10). The processing portion 20 determines whether the brain wave detecting apparatus acquires the brain wave signals for a given constant time or not (step S12). When the answer is “No”, the process goes back to step S10 and, when the answer is “Yes”, the processing portion 20 compares the brain wave signals transmitted from the brain wave detecting portions 10 with each other (step S14). Then, the processing portion 20 selects the brain wave detecting portion 10 that is to be used, according to the brain wave signals from the brain wave detecting portions 10 (step S16). For example, the processing portion 20 may select one brain wave detecting portion 10 that transmits the strongest brain wave signal among the plurality of the brain wave detecting portions 10. In another example, the processing portion 20 may select another brain wave detecting portion 10 that transmits the most stable brain wave signal for the given time. The processing portion 20 writes information about the selected brain wave detecting portion 10 into the memory 22 (step S18). Then, the initializing process is completed.

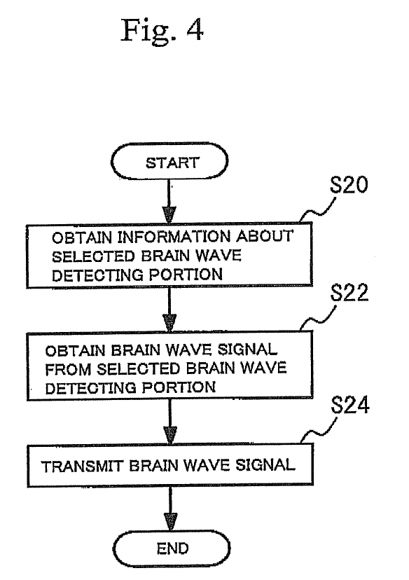

FIG. 4 is a flowchart of an operation of the processing portion 20 executed when the brain wave detecting apparatus is used after the initializing process. Referring to FIG. 4, the processing portion 20 receives the information about the brain wave detecting portion 10 selected by step S16 in FIG. 3 stored in the memory 22 (step S20). Next the processing portion 20 selects the brain wave detecting portion 10 that is to be used according to the information of the selected brain wave detecting portion 10, and receives the brain wave signal transmitted therefrom (step S22). The processing portion 20 modulates the RF signal by the information of the received brain wave signal and transits the modulate RF signal via the RF circuit 26 and the antenna 28 (step S24). The above information use for modulation may be the brain wave signal itself or information obtained by processing the brain wave signal in a given way.

The RF signal that is transmitted from the antenna 28 is received by a snooze detecting unit such as in Document 2. The snooze detecting unit determines whether the user is liable to snooze or not by the alpha wave that is one of the brain waves of the user. When the answer is yes, the snooze detecting unit issues an alert to the user so as to restrain the user from snoozing.

As to step S10 shown in FIG. 3, the processing portion 20 (selecting portion) receives the brain wave signals that are respectively transmitted from the plurality of the brain wave detecting portions 10. As to step S14 and S16, the processing portion 20 (selecting portion) selects the brain wave detecting portion 10 that is to be used from among the plurality of the brain wave detecting portions 10, based on the brain wave signals. As to step S24 shown in FIG. 4, the RF circuit 26 and the antenna 28 (transmitting portion) transmit the information relating to the brain wave signal that is transmitted from the selected brain wave detecting portion 10. With this configuration, the processing portion 20 can select the brain wave detecting portion 10 that can detect brain wave signal stably from among the plurality of the brain wave detecting portions 10. Thus, the brain wave detecting apparatus is capable of outputting the brain wave signal reliably, in spite of the condition of the brain wave detecting apparatus put on the user 50.

Next, a description is given of selection of the appropriate brain wave detecting portion 10 when the user 50 changes the way of putting on the bat 40. Referring to FIG. 2, when the user 50 puts on the hat 40 in ordinary way, a center brain-wave detecting portion 10a is the most stable to transmit the brain wave signal among the five brain wave detecting portions 10. In this case, the processing portion 20 selects the brain wave detecting portion 10a to be used.

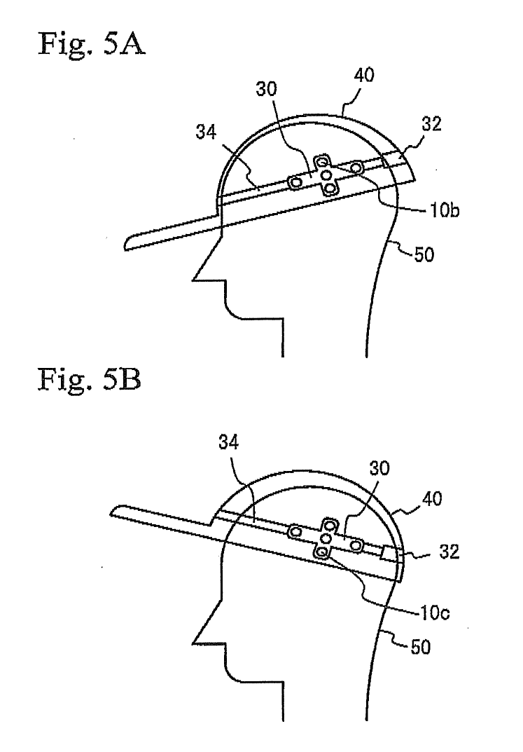

As to FIG. 5A, when the user 50 wears the hat 40 low over user's eyes, a brain wave detecting portion 10b contacts the portion where the brain wave detecting portion 10a is contacted in FIG. 2. Thus the brain wave detecting portion 10b is the most stable to transmit the brain wave signal. The processing portion 20 selects the brain wave detecting portion 10b to be used. As to FIG. 5B, when the user 50 wears the hat 40 shallowly, the processing portion 20 selects a brain wave detecting portion 10c to be used.

In this way, the brain wave is detected stably by using one of the brain wave detecting portions 10a, 10b and 10c, which are arranged in a longitudinal direction (direction to the top of the head) of the side surface of the head of the user 50, even when the user 30 changes the ways of wearing the hat according to his/her mood.

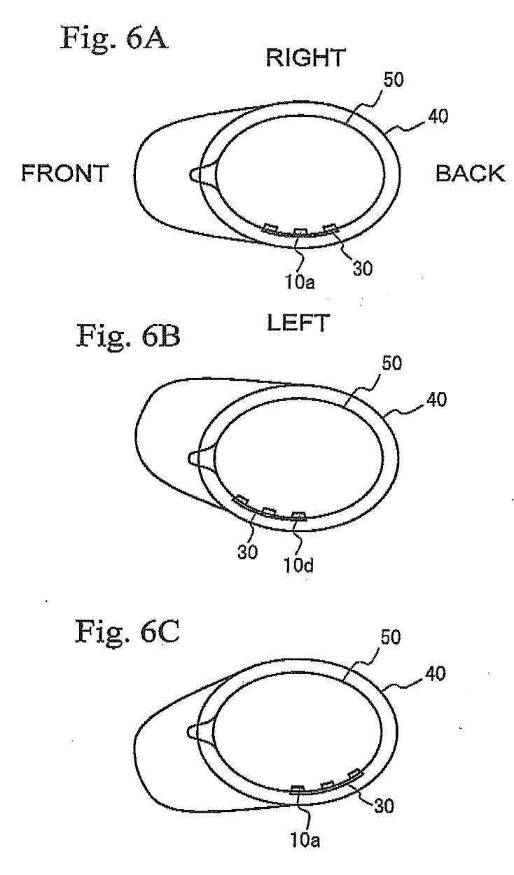

FIGS. 6A through 6C show the heads of the user 50 observed from the above. The base 30 is shown seen through the hat 40. As to FIG. 6A the brain wave detecting portion 10a is the most stable to transmit the brain wave signal among the five brain wave detecting portions 10, when the user 50 wears the hat 40 to the front of the head. In this case, the processing portion 20 selects the brain wave detecting portion 10a to be used.

As to FIG. 6B, when the user 50 wears the hat 40 with a brain thereof being slightly oriented to the right, the brain wave detecting portion 10d touches the portion where the brain wave detecting portion 10a touches in FIG. 6A. Thus, the brain wave detecting portion 10d transmits the most stable brain wave signal. Thus, the processing portion 20 selects the brain wave detecting portion 10d to be used. As to FIG. 6C, when the user 50 wears the hat 40 with the brim thereof being slightly oriented to the left, the processing portion 20 selects the brain wave detecting portion 10e to be used.

In this way, if the user 50 changes the way of wearing the hat, the brain wave signal is detected stably, since one of the brain wave detecting portions 10a, 10d and 10e, which are arranged in a lateral direction on the side surface of the head (an intersecting direction with the direction to the top of the head), can detect the brain wave.

The brain wave detecting portions 10 are arranged in the longitudinal and lateral directions on the side surface of the head so as to form a cross shape. It is thus possible to detect the brain wave stably, even when the user 50 changes the way of wearing the hat, which may be worn deeply, shallowly, or laterally inclined to the right or the left. The present invention is not limited to the exemplary arrangement of the first embodiment having three brain wave detecting portions 10 arranged in each of the vertical and lateral directions but may include an arbitrary number of brain wave detecting portions 10 in each direction.

As disclosed in Document 4, the brain wave detecting system for diagnosing has the brain wave detecting portions arranged all over the head. In contrast for an application required to simply detect the brain waves, it is enough to detect brain waves of only a part of the head. The plurality of the brain wave detecting portions 10 are arranged on the side surface of the head where the brain waves can be easily detected, so that the brain waves can be detected stably, despite the ways of wearing the hat 40. The side surface of the head on which the brain wave detecting portions 10 are arranged may be one of the right, left, front and back surfaces of the head shown in FIG. 6A. Especially, it is preferable to arrange the brain wave detecting portions 10 on one of the right, left, front and back surfaces of a bold portion of the head. Preferably, the brain wave detecting portions 10 are arranged on only the right or left surface of the head, since the user 50 comes to have a narrow view if the brain wave detecting portions 10 are arranged in front of the head.

In this way, the brain wave can be detected stably, despite the ways of wearing the hat 40, by using the brain wave detecting portions 10 that are arranged on the right or left side so as to form the cross shape in the longitudinal and the lateral directions.

It is troublesome for the user 50 to wear the brain wave detecting portions 10. However, the user can wear the brain wave detecting portions 10 naturally by wearing the hat 40 that has the plurality of brain wave detecting portions 10.

Further, the user 50 can wear the brain wave detecting portions 10 without being aware of these portions by arranging the brain wave detecting portions 10 on a driver's hat, such as a hat frequently employed in a delivery company.

The first embodiment is an exemplary snooze restraining apparatus using the brain wave signals. However, the present invention is not limited to the above snooze restraining apparatus for vehicle but may include another application for workers in offices, or for fatigue detecting. The transmission of the brain wave signals is not limited to the combination of the RF circuit 26 and the antenna 28 but may be implemented by using wires.

The present invention is not limited to the specifically disclosed embodiments, but other embodiments and variations may be made without departing from the scope of the present invention.

The present application is based on Japanese Patent Application No. 2007-150990 fled Jun. 6, 2007, the entire disclosure of which is hereby incorporated by reference.

What is claimed is:

1. A brain wave detecting apparatus comprising:
a plurality of brain wave detecting portions that are arranged on a side surface of a head and detect brain waves of the head;
a selecting portion selecting one of the plurality of brain wave detecting portions on the basis of brain wave signals transmitted from the plurality of brain wave detecting portions; and
a transmitting portion transmitting information about the brain wave signal transmitted from said one of the plurality of brain wave detecting portions.

2. The brain wave detecting apparatus as claimed in claim 1, wherein the plurality of the brain wave detecting portions are arranged on the side surface of the head in a vertical direction.

3. The brain wave detecting apparatus as claimed it claim 1, wherein the plurality of the brain wave detecting portions are arranged on the side surface of the head in a lateral direction.

4. The brain wave detecting apparatus as claimed in claim 1, wherein the plurality of the brain wave detecting portions are arranged in vertical and lateral directions on the side surface of the head so as to form a cross shape.

5. The brain wave detecting apparatus as claimed in claim 1, further comprising a hat on which the plurality of the brain wave detecting portions are arranged.
